(19) United States
(12) Patent Application Publication
(10) Pub. No.: US 2010/0119457 A1
(43) Pub. Date: May 13, 2010

(54) COMPOSITION OF LABELED AND NON-LABELED MONOCLONAL ANTIBODIES

(76) Inventors: Helmut Lenz, Tutzing (DE); Wemer Scheuer, Penzberg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/593,621

(22) PCT Filed: Mar. 27, 2008
(86) PCT No.: PCT/EP2008/002395

§ 371 Date: Sep. 29, 2009

(30) Foreign Application Priority Data
Mar. 30, 2007 (EP) 07006617.0

(51) Int. Cl. A61K 49/00; A61P 35/00

(52) U.S. Cl. 424/9.6

(57) ABSTRACT

This invention relates to a composition of labeled and non-labeled monoclonal antibodies directed to a human transmembrane protein for the simultaneous treatment and diagnosis of diseases which are associated with an overexpression of such a protein especially of cancer. The invention further relates to a method of first administering said composition, determine the change of labeled antibody concentration and afterwards administering the non-labeled monoclonal antibodies only such that the minimum required concentration of such non-labeled antibody for a favorable therapeutical effect is achieved and maintained in the treatment, while unfavorable side effects are minimized due to the lower systemic antibody concentration.

This invention relates to a composition of labeled and non-labeled monoclonal antibodies directed to a human transmembrane protein for the simultaneous treatment and diagnosis of diseases which are associated with an overexpression of such a protein especially of cancer. The invention further relates to a method of first administering said composition, determine the change of labeled antibody concentration and afterwards administering the non-labeled monoclonal antibodies only such that the minimum required concentration of such non-labeled antibody for a favorable therapeutical effect is achieved and maintained in the treatment, while unfavorable side effects are minimized due to the lower systemic antibody concentration.

### BACKGROUND OF THE INVENTION

### Monoclonal Antibodies in the Therapy

In an ongoing quest to improve the therapeutic arsenal against cancer, a fourth weapon other than surgery, chemotherapy and radiotherapy has emerged, i.e. targeted therapy. Targeted therapy includes, tyrosine kinase receptor inhibitors (small molecule inhibitors like imatinib, gefitinib, erlotinib), proteasome inhibitors (bortezomib), biological response modifiers (denileukin diftitox) and monoclonal antibodies (MAbs). The remarkable specificity of MAbs as targeted therapy makes them promising agents for human therapy. Not only can MAbs be used therapeutically to protect against disease, they can also be used to diagnose a variety of illnesses, measure serum protein and drug levels, type tissue and blood and identify infectious agents and specific cells involved in immune response. About a quarter of all biotech drugs in development are MAbs, and about 30 products are in use or being investigated. A majority of the MAbs are used for the treatment of cancer. (Gupta, N., et al., Indian Journal of Pharmacology 38 (2006) 390-396; Funaro, A., et al., Biotechnology Advances 18 (2000) 385-401; Suemitsu, N; et al., Immunology Frontier 9 (1999) 231-236).

### Labeled Monoclonal Antibodies and In-Vivo Imaging

Several in vivo imaging methods are available for the quantification of therapeutic antibodies in tumor tissue usually based on labeled derivatives of the antibodies. Said labeled antibodies usually include antibodies labeled with radiolabels such as, e.g. ¹²⁴I, ¹¹¹In, ⁶⁴Cu, and others, for use in positron emission tomography. (PET) (see e.g. Robinson, M. K., et al., Cancer Res 65 (2005) 1471-1478; Lawrentschuk, N., et al., BJU International 97 (2006) 916-922; Olafsen, T., et al., Cancer Research 65 (2005) 5907-5916; and Trotter, D. E., et al., Journal of Nuclear Medicine 45 (2004) 1237-1244), ¹²³ I, ¹²⁵I, and ^{99m}Tc and others for use in single photon emission computed tomography (SPECT) (see e.g. Orlova, A., et al., Journal of Nuclear Medicine 47 (2006) 512-519; Dietlein, M., et al., European Journal of Haematology 74 (2005) 348-352).

Also nonradioactive labels are known for in-vivo imaging techniques, e.g. near-infrared (NIR) fluorescence labels, activatable dyes, and engodogenous reporter groups (fluorescent proteins like GFP-like proteins, and bioluminescent imaging) (Licha, K., et al., Adv Drug Deliv Rev, 57 (2005) 1087-1108). Especially NIR fluorescence imaging can be used for the quantification of therapeutic antibodies in tumor tissue. Advantages of near infrared imaging over other currently used clinical imaging techniques include the following: potential for simultaneous use of multiple, distinguishable probes (important in molecular imaging); high temporal resolution (important in functional imaging); high spatial resolution (important in vivo microscopy); and safety (no ionizing radiation).

In NIR fluorescence imaging, filtered light or a laser with a defined bandwidth is used as a source of excitation light. The excitation light travels through body tissues. When it encounters a near infrared fluorescent molecule (“contrast agent”), the excitation light is absorbed.

The fluorescent molecule then emits light (fluorescence) spectrally distinguishable (slightly longer wavelength) from the excitation light. Despite good penetration of biological tissues by near infrared light, conventional near infrared fluorescence probes are subject to many of the same limitations encountered with other contrast agents, including low target/background ratios.

Near infrared wavelengths (approximately 640-1300 nm) have been used in optical imaging of internal tissues, because near infrared radiation exhibits tissue penetration of up to 6-8 centimeters. See, e.g., Wyatt, J. S., Phil. Trans. R. Soc. B 352 (1997) 697-700; Tromberg, B. J., et al., Phil. Trans. R. Soc. London B 352 (1997) 661-667.

The exact amounts of the antibody-label conjugates used for in vivo imaging depends on the different characteristics and aspects of the labels used, e.g. for NIR fluorescence labels the quantum yield of the label is one of the criteria for the amount of label or labeled antibody used (see e.g. WO 2006/072580).

### Administration and Monitoring of Non-Labeled Monoclonal Antibodies in the Therapy

Factors affecting the successful therapy of malignant diseases include the antibody dose used and the schedule of administration, the half-life and fast blood clearance of the antibodies, the presence of circulating antigen, poor tumor penetration of the high/mol.-wt. monoclonal antibody (mAb) and the way in which these molecules are catabolized. At present, there is a lack of knowledge about many aspects of the physiological function and metabolism of antibodies. (Iznaga-Escobar, N., et al, Meth. Find. Exp. Clin. Pharm. (2004) 26(2) 123-127).

The dosing and administration patterns of antibodies in the therapy of malignant diseases is usually based on the serum pharmacokinetic properties of such antibodies, like serum half-life, AUC at different dosages, the blood clearance and others (Iznaga-Escobar, N., et al., Meth. Find. Exp. Clin. Pharm. (2004) 26(2) 123-127; Lobo, E. D., et al., J. Pharm Sci. 93 (2004) 2645-2668; Tabrizi, M. A., et al., Drug Discovery Today 11 (2006) 81-88).

For example, in tumor treatment, high serum levels of antibodies targeting solid tumors are actually thought to be basic requirements for subsequent therapeutic evaluation. This evaluation is often difficult as the serum levels often differ enormously from patient to patient. However, a therapeutic monoclonal antibody, which binds in the most optimal way to its relevant target, will have a faster serum clearance (target correlated/mediated clearance) compared to an antibody with lower affinity to the relevant target. This may be one reason why plasma levels of therapeutic antibodies do not always correlate with concentration of antibodies in tumor tissue (Clarke, K., et al., Cancer Res. 60 (2000) 4804-4811; Chrastina, A., et al., Int J Cancer 105 (2003) 873-881; Lub-de Hooge, M. N., et al., Brit J. Pharmacol. 143 (2004) 99-106; Robinson, M. K., et al., Cancer Res 65 (2005) 1471-1478; Kenanova, V., et al., Cancer Res. 65 (2005) 622-631; reviewed in Batra, S. K., et al., Curr Opin Biotechnol. 6 (2002) 603-608. Consequently, high serum levels of a therapeutic antibody (especially antibodies against tumor associated antigens) may indicate diminished binding to the target. Furthermore, in case a therapeutic antibody is overdosed (above the tumor saturation dose) free antibody can bind to lower affinity epitopes (or to Fc receptors on immune effector cells) and this may lead to unwanted side effects like e.g. cardiac failure in anti-HER2-antibody treatment due to the HER2 inhibition on cardiac myocytes (Grazette, L. P; et al.; J Am Coll Card (2004), 44(11), 2231-8; Negro, A., Recent Progress in Hormone Research 59 (2004) 1-12; Negro, A., et al., PNAS 103 (2006) 15889-15893). Therefore, measurements of serum levels alone together with the associated serum half-life may be misleading when the most appropriate administration pattern has to be defined. Thus, quantitative information regarding the tumor saturation dose is an important issue.

### Side Effects of Labeled Antibodies

Monoclonal antibodies labeled with radioactive labels have one big drawback due to the cellular damage such labels can cause in healthy cells. Particularly, when these radioactive labeled antibodies are use for diagnosis these side effects are unwanted. Actually there exist different monoclonal antibodies covalently coupled to a nonradioactive label (Ballou, B., et al., Proceedings of SPIE—The International Society for Optical Engineering 2680 (1996) 124-131; Ballou, B., et al., Cancer detection and prevention (1998) 22 251-257; Becker, A., et al., Nature Biotechnology 19 (2001) 327-331; Montet, X., et al., Cancer Research 65 (2005) 6330-6336; Rosenthal, E, L., et al., The Laryngoscope 116 (2006) 1636-1641; Hilger, I., et al, European Radiology 14 (2004) 1124-1129; EP 1 619 501, WO 2006/072580, WO 2004/065491 and WO 2001/023005).

These conjugates were used in in-vivo imaging techniques to detect the disease site and size (e.g. of tumors or inflammations). This diagnostic applications are all intended fort the diagnosis before or after a therapy by either surgery, or chemotherapeutic agents including monoclonal antibodies. Normally these labeled monoclonal antibodies were used in diagnostic doses in which the side effects of the used non-radioactive labels play a minor role (compared to the use of radioactive labels).

However if these labeled monoclonal antibodies would be used for therapy the amount of nonradioactive label is critical due to the sometimes severe toxicities of these labels (especially of cyanine and carbocyanine dyes; see e.g. Kues, H. A.; Lutty, G. A; “Dyes can be deadly”; Laser Focus (1975) 11(5) 59-61.). It is therefore doubtful that these labeled monoclonal antibodies can be directly used as therapeutics or in therapeutic doses (see e.g Hilger, I., et al, European Radiology 14 (2004) 1124-1129) without causing unwanted side effects.

### Side Effects of Therapeutic Antibodies

As the unwanted side effects of monoclonal antibody treatment play a major role in the course of that treatment and the maximum duration of such a treatment, the gathering of sufficient information about the time dependency of the antibody concentration at the disease area (region of interest, e.g. at the tumor or inflammation site) during a first treatment with said antibody is an important issue. This would allow the adoption of the dose scheduling for consecutive treatments/administrations in such a way that unwanted overdosing is minimized and the minimum of necessary antibody concentration is used.

Also the differences in time dependency of the antibody concentration from patient to patient could be taken into account to optimize the individual dose scheduling with respect to a minimization of side effect.

### SUMMARY OF THE INVENTION

The invention comprises a pharmaceutical composition comprising
a) a monoclonal antibody binding to the extracellular domain of a human transmembrane protein and
b) said antibody covalently coupled to a NIR fluorescence label,
   in a predetermined ratio of at least 1:9 of non-labeled to labeled antibody.

Preferably the monoclonal antibody is a therapeutic monoclonal antibody.

Typical ratios of non-labeled antibody to labeled antibody are at least 1:9. In one preferred embodiment the ratio is at least 2:1, in another preferred embodiment the ratio is at least 9:1, in still another preferred embodiment the ratio is at least 19:1.

The maximum ratio is typically limited by the detection limit of the label. Thus an ideal ratio would be one with the lowest part of labeled antibody which still gives a sufficient NIR fluorescence signal or image during detection. In this way the non-labeled therapeutic monoclonal antibody would be affected least in his mode of action an therapeutic effect, while at the same time, important information about the kinetics of the labeled antibody in the region of e.g. a solid tumor can be gathered, which can be used as a base for an optimized dose interval or scheme. The ratio of non-labeled antibody to labeled antibody can be evaluated by a person skilled in the art in routine experiments. In this connection, the composition typically comprises the labeled antibody in an amount of at least 0.001 mg/kg body weight, preferably 0.01 mg/kg body weight, more preferably 0.1 mg/kg body weight. The exact amount can vary and depends e.g. on the label and its quantum yield. The amount can be defined by the skilled artisan by simple routine experiments. Thus the upper limit of the ratio also varies depending on the typical therapeutic dose and the detection limit of the label. Based on the typical dosages of monoclonal antibodies for therapeutic treatment (e.g the trastuzumab dose lays around 2 two 8 mg/kg body weight), one preferred maximum ratio is e.g. 500:1, another is 100:1, another is 50:1, still another is 20:1.

Preferably the human protein is an overexpressed human protein, more preferably an overexpressed tumor-associated protein.

Thus the invention comprises a pharmaceutical composition comprising
a) a therapeutic monoclonal antibody binding to the extracellular domain of an overexpressed tumor-associated protein, wherein the overexpression is associated with the tumor disease, and
b) said therapeutic monoclonal antibody covalently coupled to a NIR fluorescence label,
   in a predetermined ratio of at least 9:1 and at maximum 100:1 of non-labeled to labeled antibody.

This composition can be used to treat a patient with a disease which is associated to the overexpression of such human protein (e.g. cancer with an associated protein overexpression such as HER-positive breast cancer) and serves at the same to determine an optimized dose interval (for the individual patient in dependency of his drug metabolism).

The length of the dose interval is mainly determined based on two aspects. On the one hand, it has to be short enough such that the amount of the monoclonal antibody at the site of the disease is sufficient to exert an therapeutic effect, on the other hand is has to be long enough to minimize an overdosing and drug-associated side effects.

Usually the dose interval is determined by separate measurements of 1) e.g. the serum level of the monoclonal antibody and 2) the efficacy of the treatment, which are correlated afterwards. However, using this approach, the different metabolism of different patients is neglected or is lost by the forming the average of a greater group of patients. Thus the new composition comprising non-labeled and labeled therapeutic monoclonal antibodies.

Another embodiment of the invention is the use of said non-labeled a therapeutic monoclonal antibody binding to the extracellular domain of an overexpressed tumor-associated protein, wherein the overexpression is associated with the tumor disease

for the manufacture of said pharmaceutical composition comprising
a) a therapeutic monoclonal antibody binding to the extracellular domain of an overexpressed tumor-associated protein, wherein the overexpression is associated with the tumor disease, and
b) said therapeutic monoclonal antibody covalently coupled to a NIR fluorescence label,
   in a predetermined ratio of at least 9:1 and at maximum 100:1 of non-labeled to labeled antibody,
   for a first tumor treatment
   characterized in that
   a second tumor treatment with a second pharmaceutical composition comprising the non-labeled monoclonal antibody and not the labeled monoclonal antibody is administered when the signal intensity of the antibody covalently coupled to a NIR fluorescence label at the tumor site is 80% of the maximum signal intensity at the tumor site measured after the first treatment.

In another embodiment the second treatment is given when the signal intensity is 70%, in still another embodiment the signal intensity is 60%.

Another embodiment of the invention is said composition

comprising
a) a therapeutic monoclonal antibody binding to the extracellular domain of an overexpressed tumor-associated protein, wherein the overexpression is associated with the tumor disease, and
b) said therapeutic monoclonal antibody covalently coupled to a NIR fluorescence label,
   in a predetermined ratio of at least 9:1 and at maximum 100:1 of non-labeled to labeled antibody,
   for a first tumor treatment characterized in that
   a second tumor treatment with second pharmaceutical composition comprising the non-labeled monoclonal antibody and not the labeled monoclonal antibody is administered when the signal intensity of the antibody covalently coupled to a NIR fluorescence label in the region of the solid tumor is 80% of the maximum signal intensity in the region of the solid tumor measured after the first treatment.

In another embodiment the second treatment is given when the signal intensity is 70%, in still another embodiment the signal intensity is 60%.

Another embodiment of the invention is pharmaceutical composition comprising
a) a therapeutic monoclonal antibody binding to the extracellular domain of an overexpressed tumor-associated protein, wherein the overexpression is associated with the tumor disease; and
b) said therapeutic monoclonal antibody covalently coupled to a NIR fluorescence label,
   in a predetermined ratio of at least 9:1 and at maximum 100:1 of non-labeled to labeled antibody,
   for a first tumor treatment and a pharmaceutical composition comprising the non-labeled monoclonal antibody and not the labeled monoclonal antibody for a second tumor treatment.

Another embodiment of the invention is the a container comprising
a) a pharmaceutical composition comprising
b) a therapeutic monoclonal antibody binding to the extracellular domain of an overexpressed tumor-associated protein, wherein the overexpression is associated with the tumor disease; and
c) said therapeutic monoclonal antibody covalently coupled to a NIR fluorescence label,
   in a predetermined ratio of at least 9:1 and at maximum 100:1 of non-labeled to labeled antibody,
   for a first tumor treatment, and
a) a pharmaceutical composition comprising the non-labeled therapeutic monoclonal antibody binding to the extracellular domain of an overexpressed tumor-associated protein, and not said labeled therapeutic monoclonal antibody, for a second tumor treatment.

One embodiment of the invention is the use of said monoclonal antibody for the manufacture of said pharmaceutical composition for the treatment of cancer, preferably of solid tumors.

Another embodiment of the invention is the use of a non-labeled therapeutic monoclonal antibody binding to the extracellular domain of an overexpressed tumor-associated protein for the manufacture of a pharmaceutical composition for the treatment of cancer, preferably of a solid tumor, characterized in that the non-labeled monoclonal antibody is co-administered with said antibody covalently coupled to a NIR fluorescence label in a predetermined ratio of at least 9:1 and at maximum 100:1 of non-labeled to labeled antibody.

Another embodiment of the invention is the use of a non-labeled therapeutic monoclonal antibody binding to the extracellular domain of an overexpressed tumor-associated protein for the manufacture of a medicament for the treatment of a patient suffering from a solid tumor overexpressing said tumor-associated protein wherein the non-labeled antibody is co-administered with said antibody covalently coupled to a NIR fluorescence label.

In one embodiment of the invention, a NIR fluorescence image of a said patient suffering from a solid tumor overexpressing said tumor-associated protein is acquired.

In another embodiment of the invention, the NIR fluorescence signal of said antibody covalently coupled to a NIR fluorescence label in a region of the solid tumor is measured.

Another embodiment of the invention is a non-labeled therapeutic monoclonal antibody binding to the extracellular domain of an overexpressed tumor-associated protein for the treatment of a patient suffering from a solid tumor overexpressing said tumor-associated protein

wherein the non-labeled antibody is co-administered with said antibody covalently coupled to a NIR fluorescence label.

Another embodiment of the invention is a method for acquiring a NIR fluorescence image of a patient suffering from a solid tumor overexpressing a tumor-associated protein which has received a dose of the pharmaceutical composition comprising
a) a therapeutic monoclonal antibody binding to the extracellular domain of an overexpressed tumor-associated protein, wherein the overexpression is associated with the tumor disease; and
b) said therapeutic monoclonal antibody covalently coupled to a NIR fluorescence label,
   in a predetermined ratio of at least 9:1 and at maximum 100:1 of non-labeled to labeled antibody,
   wherein the NIR fluorescence signal of the labeled therapeutic monoclonal antibody binding to the extracellular domain of an overexpressed tumor-associated protein in a region of the solid tumor is measured.

Another embodiment of the invention is a method for determining the NIR fluorescence signal of a therapeutic monoclonal antibody covalently coupled to a NIR fluorescence label in a region of the solid tumor of a patient which has received a treatment with a pharmaceutical composition comprising
a) a therapeutic monoclonal antibody binding to the extracellular domain of an overexpressed tumor-associated protein, wherein the overexpression is associated with the tumor disease; and
b) said therapeutic monoclonal antibody covalently coupled to a NIR fluorescence label,
   in a predetermined ratio of at least 9:1 and at maximum 100:1 of non-labeled to labeled antibody.

Another embodiment of the invention is the use of a monoclonal antibody binding to the extracellular domain of a human transmembrane protein for the manufacture of the pharmaceutical composition for the treatment of cancer characterized in that the monoclonal antibody is co-administered with an antibody covalently coupled to a NIR fluorescence label in a predetermined ratio of at least 1:9 of non-labeled to labeled antibody.

Another embodiment of the invention is a method for determining the change of amount of the monoclonal antibody covalently coupled to a NIR fluorescence label during the treatment with a pharmaceutical composition comprising
a) a monoclonal antibody binding to the extracellular domain of a human transmembrane protein and
b) said antibody covalently coupled to a NIR fluorescence label,
   in a predetermined ratio of at least 1:9 of non-labeled to labeled antibody.

Another embodiment of the invention is a method for determining the change of amount of a monoclonal antibody covalently coupled to a NIR fluorescence label during co-administration with said non-labeled monoclonal antibody.

### DETAILED DESCRIPTION OF THE INVENTION

### 1. Definitions

The term “antibody” encompasses the various forms of antibodies including but not being limited to whole antibodies, human antibodies, humanized antibodies and genetically engineered antibodies like monoclonal antibodies, chimeric antibodies or recombinant antibodies as well as fragments of such antibodies as long as the characteristic properties according to the invention are retained.

The terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g. a transgenic mouse, having a genome comprising a human heavy chain transgene and a light human chain transgene fused to an immortalized cell.

The term “therapeutic monoclonal antibody” as used herein refers to a monoclonal antibody as defined above which specifically binds to the extracellular domain of a human transmembrane protein and which has an therapeutic effect on a disease which is associated with the expression of said human transmembrane protein, when administered to a patient. Preferably the therapeutic monoclonal antibody has an therapeutic effect of a tumor or cancer disease, which is associated with the expression, preferably the overexpression of said tumor or cancer disease. Typically such an anti-tumor therapeutic monoclonal antibody can be selected from e.g. the non-limiting group consisting of alemtuzumab, apolizumab, cetuximab, epratuzumab, galiximab, gemtuzumab, ipilimumab, labetuzumab, panitumumab, rituximab, trastuzumab, nimotuzumab, mapatumumab, matuzumab and pertuzumab, preferably trastuzumab, cetuximab, and pertuzumab.

The term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad Sci. USA 81 (1984) 6851-6855; U.S. Pat. No. 5,202,238 and U.S. Pat. No. 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric and bifunctional antibodies.

The term “human antibody”, as used herein, is intended to include antibodies having variable and constant regions derived from human germline immunoglobulin sequences. Human antibodies are well-known in the state of the art (van Dijk, M. A., and van de Winkel, J. G., Curr. Opin. in Chemical Biology 5 (2001) 368-374). Based on such technology, human antibodies against a great variety of targets can be produced. Examples of human antibodies are for example described in Kellermann, S. A., et al., Curr Opin Biotechnol. 13 (2002)593-597.

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form. The recombinant human antibodies according to the invention have been subjected to in vivo somatic hypermutation. Thus, the amino acid sequences of the VH and VL regions of the recombinant antibodies are sequences that, while derived from and related to human germline VH and VL sequences, may not naturally exist within the human antibody germline repertoire in vivo.

As used herein, “binding” or “specifically binding” refers to an antibody binding to the extracellular domain of human transmembrane protein for which the antibody is specific. Preferably the binding affinity is of about 10⁻¹¹ to 10⁻⁸ M (KD), preferably of about 10⁻¹¹ to 10⁻⁹ M.

The term “nucleic acid molecule”, as used herein, is intended to include DNA molecules and RNA molecules. A nucleic acid molecule may be single-stranded or double-stranded, but preferably is double-stranded DNA.

The “constant domains” are not involved directly in binding the antibody to an antigen but are involved in the effector functions (ADCC, complement binding, and CDC).

The “variable region” (variable region of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a (β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies according to the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat, E. A., et al., Sequences of Proteins of Immunological Interest, 5th Ed. Public Health Service, National Institutes of Health, Bethesda, Md. (1991) and/or those residues from a “hypervariable loop”.

The term “human transmembrane protein” when used herein refers to a cell membrane proteins which is anchored in the lipid bilayer of cells. The human transmembrane protein will generally comprise an “extracellular domain” as used herein, which may bind an ligand; a lipophilic transmembrane domain, a conserved intracellular domain tyrosine kinase domain, and a carboxyl-terminal signaling domain harboring several tyrosine residues which can be phosphorylated.

The human transmembrane proteins include molecules such as EGFR, HER2/neu, HER3, HER4, Ep-CAM, CEA, TRAIL, TRAIL-receptor 1, TRAIL-receptor 2, lymphotoxin-beta receptor, CCR4, CD19, CD20, CD22, CD28, CD33, CD40, CD80, CSF-1R, CTLA-4, fibroblast activation protein (FAP), hepsin, melanoma-associated chondroitin sulfate proteoglycan (MCSP), prostate-specific membrane antigen (PSMA), VEGF receptor 1, VEGF receptor 2, IGF1-R, TSLP-R, TIE-1, TIE-2, TNF-alpha, TNF like weak inducer of apoptosis (TWEAK), IL-1R, preferably EGFR, HER2/neu, CEA, CD20, or IGF1-R.

The terms “cancer” and “tumor” as used herein refer to or describe the physiological condition in mammals that is typically characterized by unregulated cell growth. Examples of cancer or tumors include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer. Preferably the cancer is a solid tumor.

The term “solid tumors” when used herein refers to tumors selected from the group of gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer.

The term “overexpressed” human transmembrane protein or “overexpression” of the human transmembrane protein is intended to indicate an abnormal level of expression of the human transmembrane protein in a cell from a disease area like a tumor or a arthritic joint within a specific tissue or organ of the patient relative to the level of expression in a normal cell from that tissue or organ. Patients having a diseases like e.g. characterized by overexpression of the human transmembrane protein can be determined by standard assays known in the art.

The terms “co-administration” or “co-administered” mean that the labeled antibody is administered simultaneously with the non-labeled antibody.

It is self-evident that the antibodies are administered to the patient in therapeutically effective amount which is the amount of the subject compound or combination that will elicit the biological or medical response of a tissue, system, animal or human that is being sought by the researcher, veterinarian, medical doctor or other clinician.

As used herein, the term “patient” preferably refers to a human in need of treatment to treat cancer, or a precancerous condition or lesion. However, the term “patient” can also refer to non-human animals, preferably mammals such as dogs, cats, horses, cows, pigs, sheep and non-human primates, among others, that are in need of treatment.

The terms “antibody covalently coupled to a label” or “labeled antibody” as used herein refer to antibodies which are conjugated to an label. Conjugation techniques have significantly matured during the past years and an excellent overview is given in Aslam, M., and Dent, A., Bioconjugation, London (1998) 216-363, and in the chapter “Macromolecule conjugation” in Tijssen, P., “Practice and theory of enzyme immunoassays” (1990) Elsevier, Amsterdam.

The term “non-labeled antibody” as used herein refers to an antibody which is not labeled.

The term “NIR” as used herein means near-infrared.

The term “region of a solid tumor” when used herein refers to a zone comprising the solid tumor. The region of a solid tumor can comprise either the whole solid tumor or only regional parts of it. The NIR fluorescence signal in the region of said solid tumor is measured, and the corresponding the NIR fluorescence images are acquired in either two-dimensional or three-dimensional form, e.g. in comparison with the surrounding non-tumorous tissue or in comparison with NIR fluorescence signals or images at different time points as a reference.

The term “in a predetermined ratio” refers to the ratio of the non-labeled antibody labeled antibody, which is determined before preparation of such composition. The ratio is chosen in connection with the intended use of such composition for e.g. the imaging of solid tumors or malignant blood cells, the imaging apparatus (e.g external or endoscopic, etc.), and depends inter alia form the quantum yield of the on the label and the antibody used.

Typical ratios of non-labeled antibody to labeled antibody are at least 1:9, preferably at least 2:1, and more preferably at least 9:1. The maximum ratio is typically limited by the detection limit of the label. In this connection, the composition typically comprises the labeled antibody in an amount of at least 0.001 mg/kg body weight, preferably 0.01 mg/kg body weight, more preferably 0.1 mg/kg body weight. The exact amount can vary and depends e.g. on the label and its quantum yield. The amount can be defined by the skilled artisan by simple routine experiments.

### 2. Detailed Description

The invention comprises a pharmaceutical composition comprising
a) a monoclonal antibody binding to the extracellular domain of a human transmembrane protein and
b) said antibody covalently coupled to a NIR fluorescence label,
   in a predetermined ratio of at least 1:9 of non-labeled to labeled antibody.

Preferably the human protein is an overexpressed human protein; and furthermore the overexpression is associated with a disease.

In a preferred embodiment, said antibody is directed against an oncological target. such as a transmembrane protein in solid tumors or circulating malignant cells. In a preferred embodiment, said antibody is directed to EGFR, HER2/neu, HER3, HER4, Ep-CAM, CEA, TRAIL, TRAIL-receptor 1, TRAIL-receptor 2, lymphotoxin-beta receptor, CCR4, CD19, CD20, CD22, CD28, CD33, CD40, CD80, CSF-1R, CTLA-4, fibroblast activation protein (FAP), hepsin, melanoma-associated chondroitin sulfate proteoglycan (MCSP), prostate-specific membrane antigen (PSMA), VEGF receptor 1, VEGF receptor 2, IGF1-R, TSLP-R, TIE-1, TIE-2, TNF-alpha, TNF like weak inducer of apoptosis (TWEAK), IL-1R, preferably EGFR, HER2/neu, CEA, CD20, or IGF1-R.

Preferably said antibody is an anti-HER2 antibody, preferably trastuzumab or pertuzumab.

Preferably said antibody is an anti-EGFR antibody, preferably cetuximab nimotuzumab, or matuzumab. Preferably said antibody is an anti-IGF1R antibody.

In one embodiment of the invention the pharmaceutical composition is characterized in that the antibody is selected from the group of:

alemtuzumab, apolizumab, cetuximab, epratuzumab, galiximabgemtuzumab, ipilimumab, labetuzumab, panitumumab, rituximab, trastuzumab, nimotuzumab, mapatumumab, matuzumab and pertuzumab, preferably trastuzumab, cetuximab, and pertuzumab.

The composition typically comprises the antibody covalently coupled to the label an amount of at least 0.001 mg/kg body weight, preferably 0.01 mg/kg body weight, more preferably 0.1 mg/kg body weight. The exact amount can vary and depends e.g. on the label and his quantum yield. The amount can be defined by the skilled artisan by simple routine experiments.

Said antibody is labeled with a near infrared (NIR) fluorescence label suitable for the measurement of the tumor concentration using NIR florescence imaging.

“Measurement” or “determining” of the NIR fluorescence signal in a region the solid tumor is performed after administration of the labeled antibody to the patient. Or, if the composition according to the invention is used, after the administration of the composition of the non-labeled antibody and the labeled antibody to the patient. The measurement can be performed on defined time points after administration, e.g., 1 day, 2 days or 3 or even more days or any other time point appropriate for acquiring a comparable NIR fluorescence signal or image in a region the solid tumor. The duration of the measurement or the time point after administration can be adjusted by a person skilled in the art in a way to get an appropriate NIR fluorescence signal or image.

For the NIR fluorescence measurement different devices and techniques can be used, e.g. for external solid tumors like breast tumors, a SoftScan® apparatus from ART Advanced Research Technologies Inc. (http://www.art.ca/en/products/softscan.html) is suitable (Intes X, Acad. Radiol. 12 (2005) 934-947) For internal disease areas, like colorectal or lung cancer endoscopic techniques or a combination of microsurgery-endoscopy can be used.

NIR fluorescence labels with excitation and emission wavelengths in the near infrared spectrum are used, i.e., 640-1300 nm preferably 640-1200 nm, and more preferably 640-900 nm. Use of this portion of the electromagnetic spectrum maximizes tissue penetration and minimizes absorption by physiologically abundant absorbers such as hemoglobin (<650 nm) and water (>1200 nm). Ideal near infrared fluorochromes for in vivo use exhibit:

(1) narrow spectral characteristics,
(2) high sensitivity (quantum yield),
(3) biocompatibility, and
(4) decoupled absorption and excitation spectra.

Various near infrared (NIR) fluorescence labels are commercially available and can be used to prepare probes according to this invention. Exemplary NIRF labels include the following: Cy5.5, Cy5 and Cy7 (Amersham, Arlington Hts., IL; IRD41 and IRD700 (LI-COR, Lincoln, Nebr.); NIR-1, (Dejindo, Kumamoto, Japan); LaJolla Blue (Diatron, Miami, Fla.); indocyanine green (ICG) and its analogs (Licha, K., et al., SPIE—The International Society for Optical Engineering 2927 (1996) 192-198; Ito, S., et al., U.S. Pat. No. 5,968,479); indotricarbocyanine (ITC; WO 98/47538); and chelated lanthanide compounds. Fluorescent lanthanide metals include europium and terbium. Fluorescence properties of lanthanides are described in Lackowicz, J. R., Principles of Fluorescence Spectroscopy, 2nd Ed., Kluwa Academic, New York, (1999).

Accordingly, said antibody is preferably labeled by a NIR fluorescence label selected from the group of Cy5.5, Cy5, Cy7, IRD41, IRD700, NIR-1, LaJolla Blue, indocyanine green (ICG), indotricarbocyanine (ITC) and SF64, 5-29, 5-36 and 5-41 (from WO 2006/072580), more preferably said antibody is labeled with a NIRF label selected from the group of Cy5.5, Cy5 and Cy7.

The methods used for coupling of the NIR fluorescence labels are well known in the art. The conjugation techniques of NIR fluorescence labels to an antibody have significantly matured during the past years and an excellent overview is given in Aslam, M., and Dent, A., Bioconjugation (1998) 216-363, London, and in the chapter “Macromolecule conjugation” in Tijssen, P., “Practice and theory of enzyme immunoassays” (1990), Elsevier, Amsterdam.

Appropriate coupling chemistries are known from the above cited literature (Aslam, supra). The NIR fluorescence label, depending on which coupling moiety is present, can be reacted directly with the antibody either in an aqueous or an organic medium. The coupling moiety is a reactive group or activated group which is used for chemically coupling of the fluorochrome label to the antibody. The fluorochrome label can be either directly attached to the antibody or connected to the antibody via a spacer to form a NIR fluorescence label conjugate comprising the antibody and a NIR fluorescence label. The spacer used may be chosen or designed so as to have a suitably long in vivo persistence (half-life) inherently.

“Measurement” or “determining” of the NIR fluorescence signal in a region the solid tumor is performed after administration of the labeled antibody to the patient. Or, if the composition according to the invention is used, after the administration of the composition of the non-labeled antibody and the labeled antibody to the patient. The measurement can be performed on defined time points after administration, e.g., 1 day, 2 days or 3 or even more days or any other time point appropriate for acquiring a comparable NIR fluorescence signal or image in a region the solid tumor. The duration of the measurement or the time point after administration can be adjusted by a person skilled in the art in a way to get an appropriate NIR fluorescence signal or image. E.g. in the first week after administration the measurement can be performed daily or every two to three days, depending on the increase of the tumor concentration. In the second and the following weeks, the measurement can be preformed every two to five days, depending on the increase and the decrease of the tumor concentration of the antibody. As the increase and the decrease of the tumor concentration depends on the type of antibody, even other measurement periods maybe appropriate, e.g. one week or longer. The measurement will be adjusted in a way to detect the change of amount of labeled antibody.

For the NIR fluorescence measurement different devices and techniques can be used, e.g. for external solid tumors like breast tumors, a SoftScan® apparatus from ART Advanced Research Technologies Inc. (http://www.art.ca/en/products/softscan.html) is suitable (Intes, X., Acad. Radiol. 12 (2005) 934-947) For internal disease areas, like colorectal or lung cancer endoscopic techniques or a combination of microsurgery-endoscopy can be used.

To detect for example the amount of labeled antibody in malignant blood cells (in leukemias) a shant in combination with blood cell counting apparatus can be used to detect the amount signal per blood cell.

An imaging system for NIR fluorescence measurement useful in the practice of this invention typically includes three basic components: (1) a near infrared light source, (2) a means for separating or distinguishing fluorescence emissions from light used for fluorochrome excitation, and (3) a detection system.

The light source provides monochromatic (or substantially monochromatic) near infrared light. The light source can be a suitably filtered white light, i.e., bandpass light from a broadband source. For example, light from a 150-watt halogen lamp can be passed through a suitable bandpass filter commercially available from Omega Optical (Brattleboro, Vt.). In some embodiments, the light source is a laser. See, e.g., Boas, D. A., et al., 1994, Proc. Natl. Acad. Sci. USA 91 4887-4891; Ntziachristos, V., et al., 2000, Proc. Natl. Acad. Sci. USA 97 2767-2772; Alexander, W., 1991, J. Clin. Laser Med. Surg. 9 416-418.

A high pass filter (700 nm) can be used to separate fluorescence emissions from excitation light. A suitable high pass filter is commercially available from Omega optical.

In general, the light detection system can be viewed as including a light gathering/image forming component and a light detection/image recording component. Although the light detection system may be a single integrated device that incorporates both components, the light gathering/image forming component and light detection/image recording component will be discussed separately.

A particularly useful light gathering/image forming component is an endoscope. Endoscopic devices and techniques that have been used for in vivo optical imaging of numerous tissues and organs, including peritoneum (Gahlen, J., et al., J. Photochem. Photobiol. B 52 (1999) 131-135), ovarian cancer (Major, A. L., et al., Gynecol. Oncol. 66 (1997) 122 132), colon (Mycek, M. A., et al., Gastrointest. Endoscopy. 48 (1998)390-394; Stepp, H., et al., Endoscopy 30 (1998) 379-386) bile ducts (Izuishi, K., et al., Hepatogastroenterology 46 (1999) 804 807), stomach (Abe, S., et al., Endoscopy 32 (2000) 281-286), bladder (Kriegmair, M., et al., Urol. Int. 63 (1999) 27-31; Riedl, C. R., et al., J. Endourol. 13 755-759), and brain (Ward, J., Laser Appl. 10 (1998) 224-228) can be employed in the practice of the present invention.

Other types of light gathering components useful in the invention are catheter-based devices, including fiber optics devices. Such devices are particularly suitable for intravascular imaging. See, e.g., Tearney, G. J., et al., Science 276 (1997) 2037-2039; Boppart, S. A., et al., Proc. Natl. Acad. Sci. USA 94, 4256-4261.

Still other imaging technologies, including phased array technology (Boas, D. A., et al., Proc. Natl. Acad. Sci. 19 USA 91 (1994) 4887-4891; Chance, B., Journal Ann. NY Acad. Sci. 838 (1998) 29-45), diffuse optical tomography (Cheng, X., et al., Optics Express 3 (1998) 118-123; Siegel, A., et al., Optics Express 4 (1999) 287-298), intravital microscopy (Dellian, M., et al., Journal Br. J Cancer 82 (2000) 1513-1518; Monsky, W. L., et al., Cancer Res. 59 (1999) 4129-4135; Fukumura, et al., Cell 94 (1998) 715-725), and confocal imaging (Korlach, J., et al., Proc. Natl. Acad. Sci. USA 96 (1999) 8461-8466; Rajadhyaksha, M., et al., J. Invest. Dermatol. 104 (1995)946-952; Gonzalez, S., et al., Journal Med. 30 (1999) 337-356) can be employed in the practice of the present invention.

Any suitable light detection/image recording component, e.g., charge coupled device (CCD) systems or photographic film, can be used in the invention. The choice of light detection/image recording will depend on factors including type of light gathering/image forming component being used. Selecting suitable components, assembling them into a near infrared imaging system, and operating the system is within ordinary skill in the art.

One embodiment of the invention is the use of said monoclonal antibody for the manufacture of said pharmaceutical composition for the treatment of cancer such as solid tumors or circulating malignant cells (e.g. in leukemias) characterized in that the pharmaceutical composition comprises said antibody covalently coupled to a NIR fluorescence label in a predetermined ratio of at least 1:9 of non-labeled to labeled antibody.

Another embodiment of the invention is the use of said monoclonal antibody for the manufacture of a pharmaceutical composition for the treatment of solid tumors characterized in that the pharmaceutical composition comprises said antibody covalently coupled to a NIR fluorescence label in a predetermined ratio of at least 1:9 of non-labeled to labeled antibody.

Another embodiment of the invention is the use of a monoclonal antibody binding to the extracellular domain of a human transmembrane protein for the manufacture of the pharmaceutical composition for the treatment of cancer characterized in that the monoclonal antibody is co-administered with an antibody covalently coupled to a NIR fluorescence label in a predetermined ratio of at least 1:9 of non-labeled to labeled antibody.

Another embodiment of the invention is the use of the pharmaceutical composition comprising
a) a monoclonal antibody binding to the extracellular domain of a human transmembrane protein and
b) said antibody covalently coupled to a NIR fluorescence label,
   in a predetermined ratio of at least 1:9 of non-labeled to labeled antibody, for a first treatment and the use of the non-labeled antibody only for a second treatment.

Another embodiment of the invention is the use of said pharmaceutical composition for the treatment of cancer, preferably solid tumors.

Another embodiment of the invention is the use of a monoclonal antibody binding to the extracellular domain of a human transmembrane protein for the treatment of cancer, preferably solid tumors characterized in that the monoclonal antibody is co-administered with said antibody covalently coupled to a NIR fluorescence label in a predetermined ratio of at least 1:9 of non-labeled to labeled antibody.

Another embodiment of the invention is the use of the pharmaceutical composition comprising
a) a monoclonal antibody binding to the extracellular domain of a human transmembrane protein and
b) said antibody covalently coupled to a NIR fluorescence label,
   in a predetermined ratio of at least 1:9 of non-labeled to labeled antibody, for a first treatment and the non-labeled monoclonal antibody only for a second treatment.

Another embodiment of the invention is a method for determining the change of amount of the monoclonal antibody covalently coupled to a NIR fluorescence label during the treatment with a pharmaceutical composition comprising
a) a monoclonal antibody binding to the extracellular domain of a human transmembrane protein and
b) said antibody covalently coupled to a NIR fluorescence label,
   in a predetermined ratio of at least 1:9 of non-labeled to labeled antibody.

Such method comprises e.g. the steps of
a) measuring the NIR fluorescence intensity in a region of interest (ROI), e.g. a solid tumor, such as a solid tumor or per blood cell, at different time points starting after the treatment with the composition of non-labeled and
b) determining the change of these NIR fluorescence intensities over the time, and
c) correlating the intensities to the amount of labeled antibody in the ROI,), e.g. a solid tumor.

Another embodiment of the invention is a method for determining the change of amount of the monoclonal antibody covalently coupled to a NIR fluorescence label in the region of interest during the treatment with said pharmaceutical composition.

Another embodiment of the invention is a method for determining the change of amount of the monoclonal antibody covalently coupled to a NIR fluorescence label in the solid tumor during the treatment with said pharmaceutical composition.

Another embodiment of the invention is a method for determining the change of amount of a monoclonal antibody covalently coupled to a NIR fluorescence label during co-administration with said non-labeled monoclonal antibody.

Another embodiment of the invention is a container comprising said pharmaceutical composition comprising
a) a monoclonal antibody binding to the extracellular domain of a human transmembrane protein and
b) said antibody covalently coupled to a NIR fluorescence label,
   in a predetermined ratio of at least 1:9 of non-labeled to labeled antibody, for a first treatment of cancer, preferably of solid tumors, and a composition comprising the non-labeled monoclonal antibody alone for a second treatment.

### DESCRIPTION OF THE FIGURES

FIG. 1 Optical Imaging for the Analysis of Target Expression In Vivo:

In the H322M s.c. model a mab against IGF1R labeled with Cy5.5 was injected i.v. at a single dose of 100 microgram per mouse and NIRF signal was measured 2 (FIG. 1a) and 5 days (FIG. 1b) therafter. Acquisition time was 3 seconds. These pictures indicate that i) the tumor cells express the relevant surface molecule, ii) the mab localizes to tumor tissue and iii) the mab accumulates over time in the target tissue.

FIG. 2 Optical Imaging for Pharmacokinetic Studies of Antibodies In Vivo:

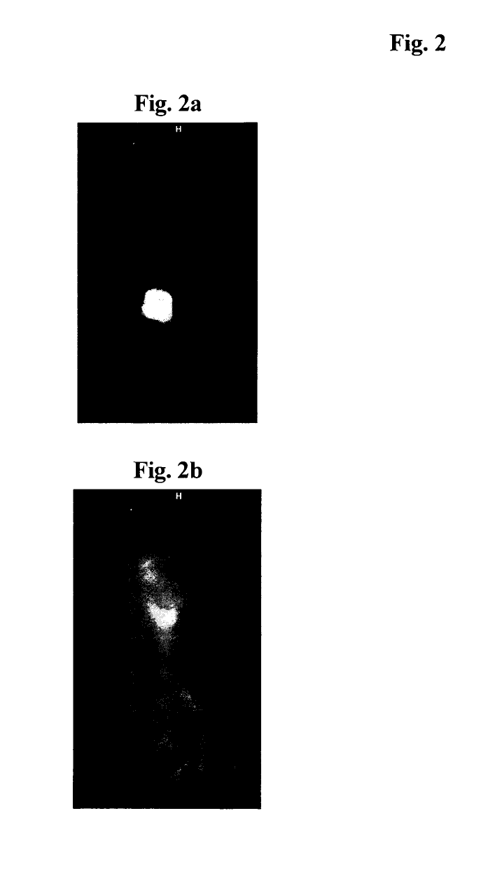

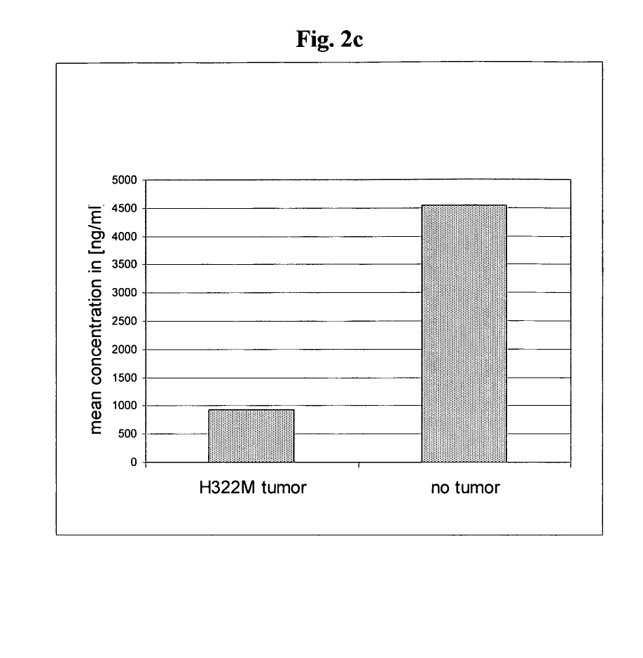

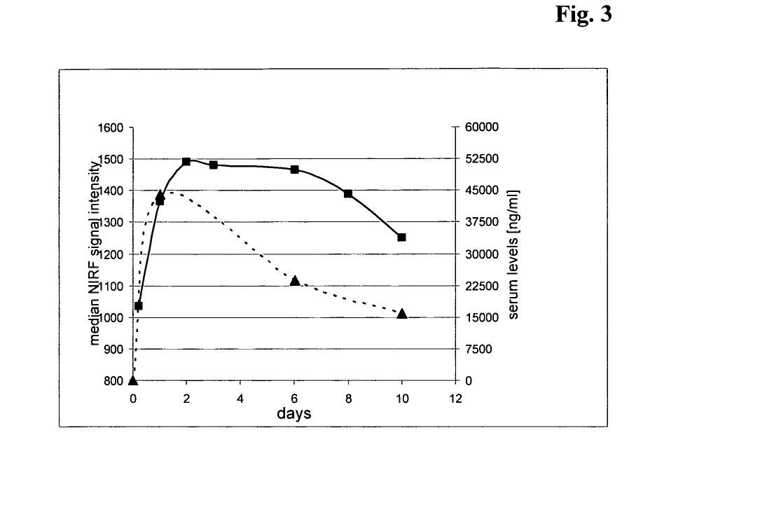

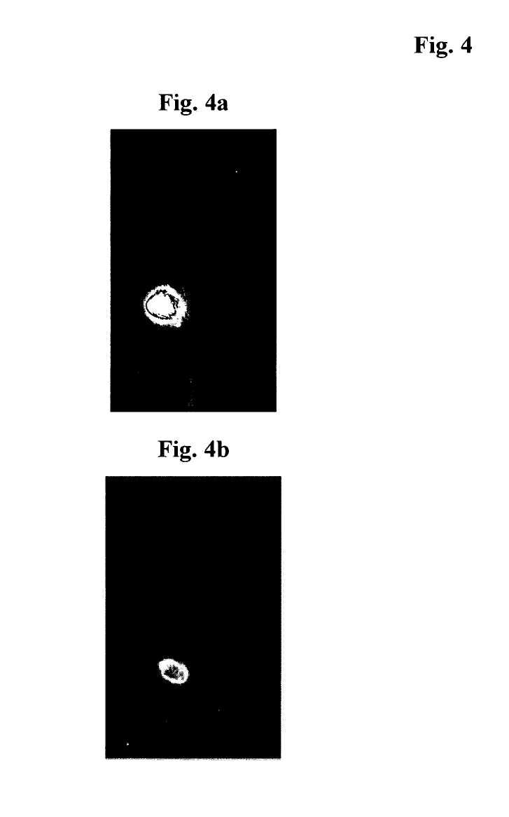

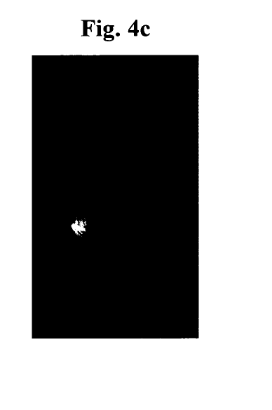

Mice with s.c. H322M tumors (FIG. 2a) and without such tumors (FIG. 2b) have been injected with 50 microgram per mouse (single dose) of an antibody against IGF1R. NIRF has been measured 4 days after application of antibody with an acquisition time of 4 seconds. FIG. 2a indicates that in tumor carrying mice the Cy5.5-labeled mab targets tumor tissue, whereas in tumor free mice the mab “lightens up” the whole mouse indicating that the mab is confined to plasma compartment (FIG. 2b) Accordingly, mab serum levels in tumor free mice (measured by Elisa) are higher compared to tumor carrying mice (FIG. 2c)

FIG. 3 Correlation of Antibody Tumor Concentrations with Serum Concentrations:

Mice carrying H460M2 tumors s.c. have been injected i.v. with a single dose (50 μg) of an antibody against IGF1R labeled with Cy5.5. At different time points (squares) therafter NIR fluorescence intensity (median NIR fluorescence (NIRF) signal intensity [arbitrary units]) was measured with an acquisition time of 4 seconds. NIR fluorescence intensity was quantified by summing up the number and signal intensities of the pixels in the region of interest (ROI) (squares and full line). In parallel, serum levels (triangles and dotted line) of said antibody against IGF1R labeled with Cy5.5 (ng/ml) was measured by ELISA. The data show, that the ratio of NIR fluorescence intensity versus serum levels increases over time, indicating that the mab accumulates in tumor tissue (FIG. 3) and that antibody concentration or the halftime of the antibody concentration in the tumor tissue is significantly longer than in serum.

FIG. 4 Detection of Relevant Tumor-Associated Antigen Using a Composition of Labeled Antibody and Non-Labeled Antibody:

The results show that the strongest NIR fluorescence signal was generated after a single injection of 50 μg Cy5-labeled anti-HER2-antibody per mouse (FIG. 4a). After a single i.v. injection of a mixture of Cy5-labeled anti-HER2-antibody and non-labeled anti-HER2-antibody at a ratio of 1 to 2 (17 μg and 33 μg) detection of Her expressing tumor is clearly detectable (FIG. 4b). FIG. 4c demonstrates that an injection of a mixture of Cy5-labeled anti-HER2-antibody and non-labeled anti-HER2-antibody at a ratio of 1 to 9 (5 μg and 45 μg) generates a significant NIR fluorescence signal. This indicates that a combination of labeled and non-labeled therapeutic antibodies in ratio 1 to 9 is feasible for application in the clinical situation.

The following examples and figures are provided to aid the understanding of the present invention, the true scope of which is set forth in the appended claims. It is understood that modifications can be made in the procedures set forth without departing from the spirit of the invention.

### EXAMPLES

### Introduction

The current study examined the in-vivo imaging of antibodies covalently coupled to a NIR fluorescence label and mixtures of antibodies covalently coupled to a NIRF-label and said antibodies without label in human xenograft models. Further aims of the study were the determination of the change in the amount of said antibody covalently coupled to a NIR fluorescence label in vivo and the comparison to the change of the corresponding serum levels.

### Cell Lines and Culture Conditions

The human breast cancer cell line KPL-4 has been established from the malignant pleural effusion of a breast cancer patient with an inflammatory skin metastasis and overexpresses ErbB family receptors. (Kurebayashi, J., et al., Br. J. Cancer 79 (1999) 707-17) Tumor cells are routinely cultured in DMEM medium (PAA Laboratories, Austria) supplemented with 10% fetal bovine serum (PAA) and 2 mM L-glutamine (Gibco) at 37° C. in a water-saturated atmosphere at 5% CO2. Culture passage is performed with trypsin/EDTA 1×(PAA) splitting twice/week. Cell passage P6 was used for in vivo study.

### Animals

SCID beige (C.B.-17) mice; age 10-12 weeks; body weight 18-20 g (Charles River, Sulzfeld, Germany) are maintained under specific-pathogen-free condition with daily cycles of 12 h light/12 h darkness according to international guidelines (GV-Solas; Felasa; TierschG). After arrival, animals are housed in the quarantine part of the animal facility for one week to get accustomed to new environment and for observation. Continuous health monitoring is carried out on regular basis. Diet food (Alltromin) and water (acidified pH 2.5-3) are provided ad libitum.

### Tumor Growth Inhibition Studies In Vivo

Tumor cells were harvested (trypsin-EDTA) from culture flasks (Greiner TriFlask) and transferred into 50 ml culture medium, washed once and resuspended in PBS. After an additional washing step with PBS and filtration (cell strainer; Falcon 100 μm) the final cell titer was adjusted to 0.75×10⁸/ml. Tumor cell suspension was carefully mixed with transfer pipette to avoid cell aggregation. Anesthesia was performed using a Stephens inhalation unit for small animals with preincubation chamber (plexiglas), individual mouse nose-mask (silicon) and Isoflurane (Pharmacia-Upjohn, Germany) in a closed circulation system. Two days before injection the fur of the animals was shaved. For intra mammary fat pad (i.m.f.p.) injection, cells were injected orthotopically at a volume of 20 μl into the right penultimate inguinal mammary fat pad of each anesthetized mouse. For the orthotopic implantation, the cell suspension was injected through the skin under the nipple. Tumor cell injection corresponds to day 1 of the experiment.

### Monitoring

Animals were controlled daily for detection of clinical symptoms of adverse effects. For monitoring throughout the experiment, the body weight of the animals was documented two times weekly.

Determination of Amount of Labeled Antibody in Tumor Tissue and of the Half-Time of that Amount in Tumor Tissue

Non-invasive measurements of near infrared signals can be accomplished by labeling proteins with appropriate dyes. E.g. different monoclonal antibodies were labeled with a Cy5 or Cy5.5 or Cy7 dyes to monitor the tumor tissue saturation of these antibodies after i.v. injection into tumor carrying mice. NIR fluorescence measurements were performed immediately after application of antibodies and at different time points therafter using the BonSAI Imaging System from Siemens Medizintechnik, Germany. Aquisition time was held constant for the complete observation period. By summing up mean intensities of the pixels in the region of interest, the area under the curve (AUC) was constructed.

Determination of Amount of Labeled Antibody in Serum of the Half-Time of that Amount in Serum

Quantification of antibody serum levels by an established ELISA was performed to correlate these results with NIR fluorescence signal intensities.

### Results

### Example 1

### Optical Imaging for the Analysis of Target Expression In Vivo

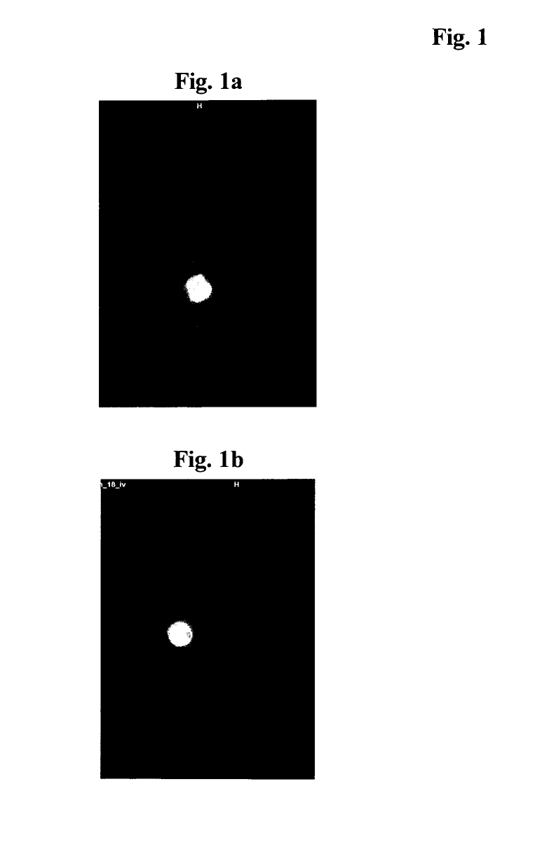

In the H322M s.c. (subcutaneous) model a mab against IGF1R labeled with Cy5.5 was injected intravenous (i.v.) at a single dose of 100 microgram per mouse and NIR fluorescence signal was measured 2 (FIG. 1a) and 5 days (FIG. 1b) therafter. Aquisition time was 3 seconds. These pictures indicate that i) the tumor cells express the relevant surface molecule, ii) the mab localizes to tumor tissue and iii) the mab accumulates over time in the target tissue.

### Example 2

### Optical Imaging for PK Studies of Antibodies In Vivo

Mice carrying s.c. H322M tumors have been injected with 50 microgram per mouse (single dose) of an antibody against IGF1R. NIR fluorescence has been measured 4 days after application of antibody with an acquisition time of 4 seconds. FIG. 2a indicates that in tumor carrying mice the Cy5.5-labeled mab targets tumor tissue, whereas in tumor free mice the mab “lightens up” the whole mouse indicating that the mab is confined to plasma compartment (FIG. 2b) Accordingly, mab serum levels in tumor free mice (measured by Elisa) are higher compared to tumor carrying mice (FIG. 2c).

### Example 3

### Correlation of NIRF Signal Intensities of with Serum Levels

Mice carrying H460M2 tumors s.c. have been injected i.v. with a single dose of an antibody against IGF1R labeled with Cy5.5. At different time points therafter NIR fluorescence was measured with an acquisition time of 4 seconds. NIR fluorescence intensity was quantified by summing up the number and signal intensities of the pixels in the region of interest (ROI). Serum levels of antibody (ng/ml) was measured by ELISA. The data show, that the ratio of NIR fluorescence versus serum levels (enrichment factor) increases over time from 31 to 79, indicating that the mab accumulates in tumor tissue (FIG. 3) and that antibody concentration in the tumor tissue is significantly longer than in serum.

### Example 4

### Detection of Relevant Tumor-Associated Antigen Using a Composition of Labeled Antibody and Non-Labeled Antibody

SCID beige mice carrying KPL-4 tumors s.c. have been injected i.v. with a single dose of a Cy5-labeled anti-HER2-antibody at a dosage of 50 μg/mouse. In addition, different group of mice have been injected with 50 μg/mouse of a mixture of labeled anti-Her2 antibody and non-labeled antibody at different ratio i) ratio of labeled to non-labelled 1 to 2 and ii) ratio of labeled to non-labeled 1 to 9). Two days thereafter fluorescence intensities in the region of interest was measured with an acquisition time of 5 seconds.

The results show that the strongest NIR fluorescence signal was generated after a single injection of 50 μg Cy5-labeled anti-HER2-antibody per mouse (FIG. 4a). After a single i.v. injection of a mixture of Cy5-labeled anti-HER2-antibody and non-labeled anti-HER2-antibody at a ratio of 1 to 2 (17 μg and 33 μg) detection of Her expressing tumor is clearly detectable (FIG. 4b). FIG. 4c demonstrates that an injection of a mixture of Cy5-labeled anti-HER2-antibody and non-labeled anti-HER2-antibody at a ratio of 1 to 9 (5 μg and 45 μg) generates a significant NIR fluorescence signal. This indicates that a combination of labeled and non-labeled therapeutic antibodies in ratio 1 to 9 is feasible for application in the clinical situation.

### Example 5

### Correlation of NIRF Signal Intensities with Serum Levels

SCID beige mice carrying KPL-4 tumors s.c. are injected i.v. with a single dose of a Cy5-labeled antibody against Her2 at a dosage of 50 μg/mouse. In addition, different group of mice are injected with 50 μg/mouse of a mixture of Cy5-labeled anti-HER2-antibody and non-labeled anti-HER2-antibody at different ratio i) ratio of labeled to non-labeled 1 to 2 and ii) ratio of labeled to non-labeled 1 to 9. At different time points therafter NIR fluorescence signals are measured with an acquisition time of 5 seconds. NIR fluorescence intensity is quantified by summing up the number and signal intensities of the pixels in the region of interest (ROI). Serum levels of antibody (ng/ml) are measured by ELISA.

### Example 6

### Reducing the Dose (and Drug Associated Side-Effects) by Prolonging the Dose Interval Based on the Antibody Concentration or the NIRF Intensity of the Labeled Antibody in the Region of the Solid Tumor

### Test Agents

Pure trastuzumab and trastuzumab labeled with Cy-5 are provided as a 25 mg/ml stock solution in Histidine-HCl, alpha-alpha Trehalose (60 mM), 0.01% Polysorb, pH 6.0. Both solutions were diluted appropriately in PBS for injections.

### Cell Lines and Culture Conditions

The human breast cancer cell line KPL-4 has been established from the malignant pleural effusion of a breast cancer patient with an inflammatory skin metastasis and overexpresses ErbB family receptors. (Kurebayashi et al. Br. J. Cancer 79 (1999) 707-17) Tumor cells are routinely cultured in DMEM medium (PAA Laboratories, Austria) supplemented with 10% fetal bovine serum (PAA) and 2 mM L-glutamine (Gibco) at 37° C. in a water-saturated atmosphere at 5% CO2. Culture passage is performed with trypsin/EDTA 1×(PAA) splitting twice/week. Cell passage P6 was used for in vivo study.

### Animals

SCID beige (C.B.-17) mice; age 10-12 weeks; body weight 18-20 g (Charles River, Sulzfeld, Germany) are maintained under specific-pathogen-free condition with daily cycles of 12 h light/12 h darkness according to international guidelines (GV-Solas; Felasa; TierschG). After arrival, animals are housed in the quarantine part of the animal facility for one week to get accustomed to new environment and for observation. Continuous health monitoring is carried out on regular basis. Diet food (Alltromin) and water (acidified pH 2.5-3) are provided ad libitum.

### Tumor Growth Inhibition Studies In Vivo

Tumor cells are harvested (trypsin-EDTA) from culture flasks (Greiner TriFlask) and transferred into 50 ml culture medium, washed once and resuspended in PBS. After an additional washing step with PBS and filtration (cell strainer; Falcon 100 μm) the final cell titer is adjusted to 0.75×10⁸/ml. Tumor cell suspension was carefully mixed with transfer pipette to avoid cell aggregation. Anesthesia is performed using a Stephens's inhalation unit for small animals with preincubation chamber (plexiglas), individual mouse nose-mask (silicon) and Isoflurane (Pharmacia-Upjohn, Germany) in a closed circulation system. Two days before injection the fur of the animals is shaved. For intra mammary fat pad (i.m.f.p.) injection, cells are injected orthotopically at a volume of 20 μl into the right penultimate inguinal mammary fat pad of each anesthetized mouse. For the orthotopic implantation, the cell suspension is injected through the skin under the nipple. Tumor cell injection corresponds to day 1 of the experiment.

### Monitoring

Animals are controlled daily for detection of clinical symptoms of adverse effects. For monitoring throughout the experiment, the body weight of the animals was documented two times weekly and the tumor volume was measured by caliper twice weekly. Primary tumor volume is calculated according to NCI protocol (TV=1/2ab2, where a and b are long and short diameters of tumor size in mm, Teicher B. Anticancer drug development guide, Humana Press, 1997, Chapter 5, page 92). Calculation values were documented as mean and standard deviation.

### Treatment of Animals

Tumor-bearing mice are randomized when the tumor volume was roughly 100 mm³ (n=10 for each group). Each group is closely matched before treatment, which began 20 days after tumor cell injection.

Group A: Vehicle group—receives 10 ml/kg PBS buffer intraperitoneally (i.p.) once weekly.

Group B: trastuzumab is administered i.p. at a loading dose of 30 mg/kg, followed by once weekly doses of 15 mg/kg (maintenance dose).

Group C: A composition of trastuzumab and Cy-5 labeled trastuzumab at a predetermined ratio of 9:1 is administered i.p. at a loading dose of 30 mg/kg.

At different time points (usually once a day) therafter NIR fluorescence signals are measured with an acquisition time of 10 seconds. NIR fluorescence intensity is quantified by summing up the number and signal intensities of the pixels in the region of the solid tumor.

First the maximum of the NIR fluorescence intensity is determined in dependency of the time. Then the time point for a first maintenance dose of 15 mg/kg only non-labeled trastuzumab is determined as the time point when the NIR fluorescence intensity has decreased by 10% compared to said maximum. The time interval between loading dose and first maintenance dose is then used as the general dosage interval between consecutive maintenance doses. The consecutive maintenance doses of 15 mg/kg only non-labeled trastuzumab are then given at this general dose.

Group D: A composition of trastuzumab and Cy-5 labeled trastuzumab at a predetermined ratio of 9:1 is administered i.p. at a loading dose of 30 mg/kg.

At different time points (usually once a day) therafter NIR fluorescence signals are measured with an acquisition time of 10 seconds. NIR fluorescence intensity is quantified by summing up the number and signal intensities of the pixels in the region of the solid tumor.

First the maximum of the NIR fluorescence intensity is determined in dependency of the time. Then the time point for a first maintenance dose of 15 mg/kg only non-labeled trastuzumab is determined as the time point when the NIR fluorescence intensity has decreased by 20% compared to said maximum. The time interval between loading dose and first maintenance dose is then used as the general dosage interval between consecutive maintenance doses. The consecutive maintenance doses of 15 mg/kg only non-labeled trastuzumab are then given at this general dose.

Group E: A composition of trastuzumab and Cy-5 labeled trastuzumab at a predetermined ratio of 9:1 is administered i.p. at a loading dose of 30 mg/kg.

At different time points (usually once a day) therafter NIR fluorescence signals are measured with an acquisition time of 10 seconds. NIR fluorescence intensity is quantified by summing up the number and signal intensities of the pixels in the region of the solid tumor.

First the maximum of the NIR fluorescence intensity is determined in dependency of the time. Then the time point for a first maintenance dose of 15 mg/kg only non-labeled trastuzumab is determined as the time point when the NIR fluorescence intensity has decreased by 30% compared to said maximum. The time interval between loading dose and first maintenance dose is then used as the general dosage interval between consecutive maintenance doses. The consecutive maintenance doses of 15 mg/kg only non-labeled trastuzumab are then given at this general dose.

Then the treatment response of Group B and Groups C to is compared, to select an optimized, prolonged dosage interval wherein the treatment response is comparable to that of Group B (in spite the lower dosage, which presumably causes less drug-associated side effects).

1. A pharmaceutical composition comprising a first amount and a second amount of a monoclonal antibody which binds to the extracellular domain of a human transmembrane protein, wherein:
said antibody in said first amount is non-labeled; and
said antibody in said second amount is covalently coupled to a NIR fluorescence label;
and wherein said first amount of said antibody is present in said composition in an amount of at least 1:9 with respect to the second amount of said antibody.

2. A pharmaceutical composition according to claim 1 wherein:
said antibody is a therapeutic monoclonal antibody which binds to the extracellular domain of an overexpressed tumor-associated protein, and wherein the overexpression of said protein is associated with tumor disease; and
wherein said first amount of said antibody is present in said composition in an amount of
at least 9:1 and at maximum 100:1 with respect to the second amount of said antibody.

3. A pharmaceutical composition according to claim 2, characterized in that said tumor-associated protein is selected from the group consisting of: EGFR, HER2/neu, HER3, HER4, Ep-CAM, CEA, TRAIL, TRAIL-receptor 1, TRAIL-receptor 2, lymphotoxin-beta receptor, CCR4, CD19, CD20, CD22, CD28, CD33, CD40, CD80, CSF-1R, CTLA-4, fibroblast activation protein (FAP), hepsin, melanoma-associated chondroitin sulfate proteoglycan (MCSP), prostate-specific membrane antigen (PSMA), VEGF receptor 1, VEGF receptor 2, IGF1-R, TSLP-R, TIE-1, TIE-2, TNF-alpha, TNF like weak inducer of apoptosis (TWEAK), IL-1R, EGFR, HER2/neu, CEA, CD20, and IGF1-R.

4-8. (canceled)

9. A kit comprising:
a) a pharmaceutical composition according to claim 2 for a first tumor treatment; and
b) a pharmaceutical composition comprising said antibody only in non-labeled form for a second tumor treatment.

10-16. (canceled)

17. The pharmaceutical composition according to claim 2, characterized in that said tumor-associated protein is selected from the group consisting of: EGFR, HER2/neu, CEA, CD20, and IGF1-R.
